(19) United States
(12) Patent Application Publication
(10) Pub. No.: US 2017/0065500 A1
(43) Pub. Date: Mar. 9, 2017

(54) Activation of Hydrogen Peroxide

(71) Applicant: Revlon Consumer Products Corporation, New York, NY (US)

(72) Inventors: Lorenzo Delpani, Bernardsville, NJ (US); Heng Cai, Skillman, NJ (US)

(21) Appl. No.: 14/849,078

(22) Filed: Sep. 9, 2015

(51) Int. Cl. A61K 8/22; B65D 51/24; A61Q 15/00

(52) U.S. Cl. CPC A61K 8/22; A61Q 15/00; B65D 51/245; A61K 2800/30; A61K 2800/81

(57) ABSTRACT

The present invention is directed to an antiperspirant/deodorant composition comprising: hydrogen peroxide, wherein said composition is essentially free of aluminum cations or zirconium cations, and the composition is exposed to a light source having a wavelength range of >220 nm and <405 nm.

### FIELD OF INVENTION

Provided herein are antiperspirant/deodorant compositions and methods of reducing perspiration and/or odor. More particularly, the present teachings relate to new and improved antiperspirant/deodorant compositions that provide a reduced level of aluminum and/or zirconium, or even no aluminum and/or zirconium, compared to known antiperspirant/deodorants.

### BACKGROUND OF THE INVENTION

Antiperspirant products typically use aluminum- and zirconium-based salts as the active antiperspirant ingredient to control perspiration and malodor. However, many consumers may hold a negative view of their use due to health and environmental concerns. Moreover, the high concentrations of the active antiperspirant ingredients can cause irreversible staining of clothing.

A homeopathic approach eliminates aluminum- and zirconium-based salts in favor of antiperspirant actives from plant origin, such as salvia officinalis oil. While these alternative compositions have met with mixed success, they are generally regarded to be much less effective in controlling perspiration than conventional compositions. Another solution to the problem is to abandon perspiration control, and instead address malodor that can arise from the perspiration, for example, by using baking soda products. However, homeopathic responses are not adequate for many people, who must reduce or eliminate perspiration using more effective means because their preferences and/or lifestyles do not permit uncontrolled sweating, or for other reasons.

Despite the concurrent advancements in antiperspirant technology, effective antiperspirant/deodorant compositions having a significantly reduced, or even a zero level of aluminum- and/or zirconium-based salts are not known, but would be enthusiastically adopted by consumers.

Accordingly, there is a need for new antiperspirant/deodorant compositions that can reduce the high concentrations of conventional antiperspirant and deodorant ingredients while maintaining antiperspirant/deodorant efficacy. That can reduce the high concentrations of conventional antiperspirant and deodorant ingredients while maintaining antiperspirant/deodorant efficacy.

An alternative to conventional antiperspirant products is the use of photochemical reactions with hydrogen peroxide.

### SUMMARY OF THE INVENTION

The present invention is directed to an antiperspirant/deodorant composition comprising: hydrogen peroxide, wherein said composition is essentially free of aluminum cations or zirconium cations, and the composition is exposed to a light source having a wavelength range of >220 nm and <405 nm.

In an embodiment the percentage of perspiration reduction provided by said composition is greater than a placebo control absent said hydrogen peroxide exposed to a light source having a wavelength range of >220 nm and <405 nm., aluminum cations, and zirconium cations.

In an embodiment the composition further comprises: an antiperspirant active, absorbent, deodorant agent, antimicrobial, dye or colorant, emollient, moisturizer, fragrance, perfume, volatile oil, emulsifier, humectant, diluent, bulking agent, distributing agents, rheology agent, glyceride oil, silica, hair growth inhibitor, pharmaceutical active, UV active, preservative, surfactant, solvent, wash-off aid, and combinations thereof.

The present invention is also directed to a method of activating hydrogen peroxide with a light source having a wavelength range of >220 nm and <405 nm in an antiperspirant/deodorant composition, wherein said composition is essentially free of aluminum cations or zirconium cations.

In an embodiment the light source is a light emitting diode.

In an embodiment the light emitting diode is contained in the cap or body of an antiperspirant container.

In an embodiment the hydrogen peroxide is encapsulated in silica dimethicone silylate.

In an embodiment the antiperspirant composition is in a form selected from the group consisting of solid, soft solid, roll-on and aerosol.

The present invention is also directed to a container comprising a base for holding an antiperspirant/deodorant composition and a cap covering the base wherein the cap contains a light source on the internal side of the cap and the external side of the cap contains a switch controlling the light source.

### BRIEF DESCRIPTION OF THE DRAWINGS

FIG. 1 is a photo of a quartz cuvette containing an encapsulated hydrogen peroxide solution radiated with UV light of various wavelengths. The top of the cuvette is covered with a balloon.

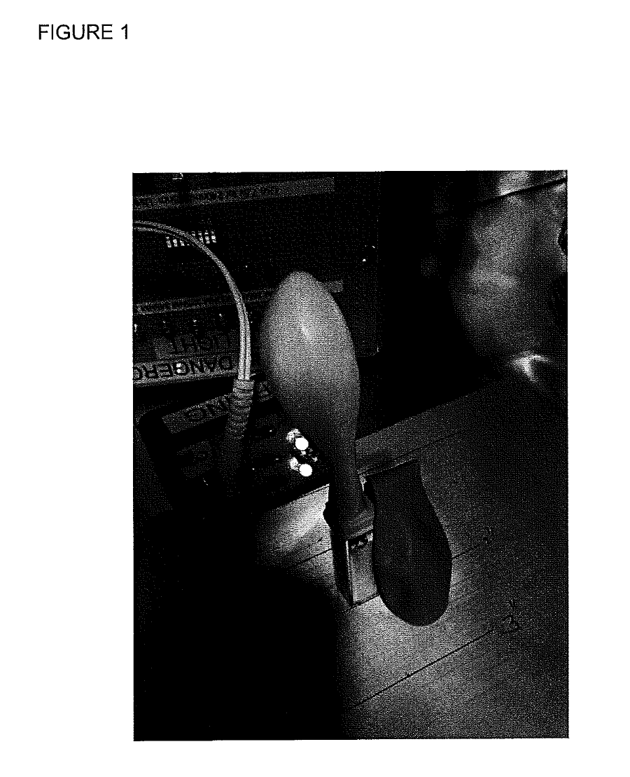

FIG. 2 is a photo of the UV light source in the on position in the cap of a container holding the claimed composition.

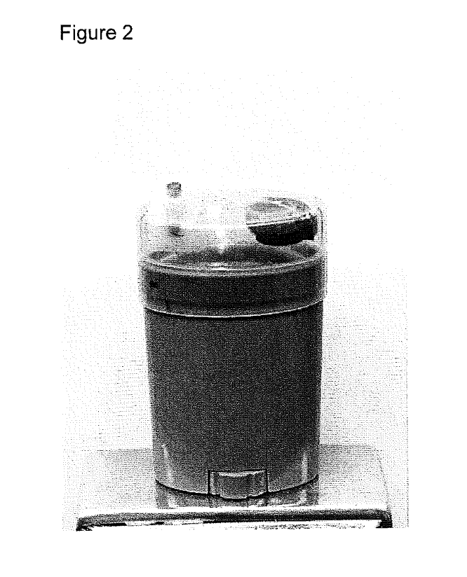

### DETAILED DESCRIPTION OF THE INVENTION

The present invention is directed to an antiperspirant/deodorant composition comprising: hydrogen peroxide, wherein said composition is essentially free of aluminum cations or zirconium cations, and the composition is exposed to a light source having a wavelength range of >220 nm and <405 nm.

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

For compositions described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

Where an element or component is said to be included in and/or selected from a list of recited elements or components, it should be understood that the element or component can be anyone of the recited elements or components, or can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” “having,” “contain,” “contains,” or “containing” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a .+0.10% variation from the nominal value unless otherwise indicated or inferred.

The term “antiperspirant/deodorant composition” refers to any composition that reduces perspiration. The term “antiperspirant/deodorant composition” includes compositions that reduce perspiration, as well as those compositions that reduce perspiration and any malodor associated with perspiration (e.g., due to bacterial growth).

Effective antiperspirant/deodorant compositions have been discovered that relieve the formulary burden of high concentrations of aluminum cations and/or zirconium cations. The invention herein described is essentially free of aluminum cations and/or zirconium cations. Within the context of the invention, the term “essentially free” refers to a concentration of aluminum cations and/or zirconium cations such that a similar composition without the hydrogen peroxide exposed to a light source having a wavelength range of >220 nm and <405 nm does not provide antiperspirant efficacy as defined by the U.S. Food and Drug Administration. Thus, the term “essentially free” is not restricted to mean “zero” or “almost zero” aluminum cations or zirconium cations, although these concepts are included in what is meant. More broadly, “essentially free” refers to a level of aluminum cations and/or zirconium cations in a similar compositions without the aforementioned hydrogen peroxide exposed to a light source having a wavelength range of >220 nm and <405 nm such that a 20% reduction in sweat production in about 50% of a given population is not attained. To illustrate this concept, consider a composition containing 3% aluminum cations and/or zirconium cations and no aforementioned hydrogen peroxide exposed to a light source having a wavelength range of >220 nm and <405 nm. If that composition does not provide at least a 20% reduction in sweat production in about 50% of a given population, then that composition is considered to be “essentially free” of aluminum cations and/or zirconium cations. Of course, analogous compositions with less than 3% aluminum cations and/or zirconium cations also are considered to be “essentially free” of those cations.

Contrary to expectations, it has been discovered that antiperspirant/deodorant compositions can effectively control perspiration without exposing the user to levels of aluminum cations or zirconium cations found in commercial products.

The cosmetic compositions of the present invention may also include one or more optional ingredients. Examples of the optional ingredients include but are not limited to lubricants, preservatives, perfumes, and colorants, oils, and waxes. The optional ingredients should be chemically inert with respect to each other, and with respect to the hydrogen peroxide or the silica encapsulating the hydrogen peroxide.

When an aqueous carrier is used, the cosmetic composition may comprise about 30 to about 90 wt % of water. Preferably, the composition comprises about 55 to about 65 wt % of distilled water. More preferably, about 10 to about 40 wt % of water, and even more preferably, about 15 to about 30 weight wt % of water. It should be understood however, that the water in the composition can be totally or partly eliminated by the use of non-aqueous or partially aqueous carriers.

Lists of carrier material and optional ingredients, which are well known in the art, are disclosed, for example, in “Cosmetics: Science and Technology,” edited by M. S. Balsam and E. Sagarin, 2nd Edition, 1972, Wiley Pub. Co.; “The Chemistry and Manufacture of Cosmetics” by M. G. DeNavasse; and “Harry's Cosmeticology,” J. B. Wilkinson et al., 7th Edition, 1982, Chem. Pub. Co.; the disclosures of each of the above being incorporated herein by reference.

As used herein, “about” or “approximately” generally means within 20 percent, preferably within 10 percent, and more preferably within 5 percent of a given value or range.

As discussed in this section, the antiperspirant/deodorant compositions may include additional ingredient(s) that may assist product manufacturing, delivery, performance, and/or perceived sensory qualities. These additional ingredient(s) typically may be selected from those compounds typically known and used in personal care products, particularly skin care. For convenience, these compounds may be considered to belong to one or more of the following categories: antiperspirant actives, absorbents, deodorant agents, antimicrobials, dyes or colorants, emollients, moisturizers, fragrances, perfumes, volatile oils, emulsifiers, humectants, diluents, bulking agents, distributing agents, rheology agents, glyceride oils, silicas, hair growth inhibitors, pharmaceuticals actives, UV actives, preservatives, surfactants, solvents (particularly non-aqueous solvents), wash-off aids. One skilled in the art recognizes that many of these materials serve multiple purposes. Many times the antiperspirant/deodorant composition will comprise more than one additional ingredient, and may be a blend of multiple additional ingredients.

In one aspect, the additional ingredients include those known in the field of antiperspirants and/or/deodorants, including the disclosures provided by U.S. Patent Application 2010/0322876, U.S. Pat. No. 6,616,921, and international patent application WO 2010/072625. These documents are hereby incorporated in their entirety by reference.

Active antiperspirant traditionally used in the antiperspirant/deodorant compositions can include any compound, composition or other material having antiperspirant activity. Active antiperspirant ingredients include astringent metallic salts, especially the inorganic salts of aluminum, zirconium, and zinc, as well as their mixtures. Depending on the product form and intended use, aluminum and zirconium salts may be used, such as aluminum halides, aluminum chlorohydrate, aluminum hydroxyhalides, zirconyl oxyhalides, zirconyl hydroxyhalides, and mixtures thereof.

Additional active antiperspirant ingredients that may be included in the formation include: aluminum chloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, and aluminum zirconium octachlorohydrate.

The antiperspirant/deodorant compositions of the present teachings can be formulated as any known or otherwise effective product form for providing topical application of an active antiperspirant ingredient and/or an active deodorant ingredient to the desired area of the skin. These compositions may be used in the axilla (i.e., arm pit), or on the body (e.g., back, neck, chest, feet, hands) where needed. The product form may be tailored to suit the needs of these application areas. Representative product forms include solid and solid-like forms (e.g., sticks, waxes, powders), liquids (e.g., aerosol sprays, pump sprays, mist sprays, roll-ons, wipes), and semi-solids (e.g., gels, creams, soft solids, lotions).

The antiperspirant/deodorant compositions of the present teachings can be prepared by any known or otherwise effective technique, suitable for providing an antiperspirant/deodorant composition of the desired form and having the essential materials described herein.

The antiperspirant salts used in the composition of the invention are solubilized in the water. While preferably the antiperspirant salts are completely dissolved in the water, in some cases small amounts of salts may not be dissolved, i.e. may remain in the crystalline or suspension form.

While the antiperspirant/deodorant compositions may or may not comprise aluminum cations nor zirconium cations. The inclusion of antiperspirant actives from natural sources may be an attractive option for all-natural products. These natural actives may be sourced from plant or inorganic material, and include compounds such as: salvia officinalis oil, psyllium, sage extract, lavender oil, rosemary oil, tea tree oil, lemon juice, lemon oil, lime juice, lime oil, baking soda (sodium bicarbonate), cornstarch, green clay, coconut oil, witch hazel extract, and oak gall extract.

It may be desired to incorporate into the antiperspirant composition one or more deodorant actives instead of the antiperspirant actives. The deodorant actives should be soluble in the aqueous single phase composition, or water dispersible carrier, such as triclosan encapsulated in cyclodextrin, which may be purchased from Lipo, Inc. Examples of suitable deodorant actives include fragrances, ammonium phenolsulfonate, benzalkonium chloride, benzethonium chloride, bromochlorophene, cetylpyridinium chloride, chlorophyllin-copper complex, chlorothymol, chloroxylenol, cloflucarban, dequalinium chloride, dichlorophene, dichloro-m-xylenol, disodium dihydroxyethyl sulfosuccinylundecylenate, domiphen bromide, hexachlorophene, lauryl pyridinium chloride, methylbenzethonium chloride, phenol, sodium bicarbonate, sodium phenolsulfonate, triclocarbone, triclosan, zinc phenolsulfonate, zinc ricinoleate, and mixtures thereof.

The plurality of particles comprising silica complexed with hydrogen peroxide. A non-limiting example of how these particles are used may be an aqueous solution of hydrogen peroxide impregnated into the silica. The solution is irradiated with a UV light source found in the cap of the product container. The time of irradiation can be determined by a switch on the cap that turns the light off. The irradiated solution is applied to a basic surface such as human skin.

### Examples

Table 1 below demonstrates that hydrogen peroxide is an effective against bacteria associated with the need for antiperspirant and deodorant. The bacterial (organism) was plated on a standard agar petri dish and allowed to grow. The colonies were treated with hydrogen peroxide alone and with UV light for 15 minutes. The light growth means light growth is visable but not as fully developed colonies. Heavy growth was fully developed colonies to numerous to count.

TABLE 1

| | | | 0.7% | | | | |
|---|---|---|---|---|---|---|---|
| | | Control | Hydrogen | | | Hydrogen | |
| | Control - | With | Peroxide | 10% | 1% Powder | peroxide + | 265 nm |
| | no light | 265 nmUV | Liquid - | antiperspirant | Hydrogen Peroxide + | 265 nm | UV |
| organism | exposure | light | no light | with cations | Cyclopentasiloxane | UV light | light |
| C. xerosis | Heavy | Heavy | 3 | No growth | Light growth | No | Light |
| | growth | growth | colonies | | | growth | growt |
| S. epidermidis | | Heavy | | Light growth | Light growth | Very | |
| | | growth | | | | Light | |
| | | | | | | growth | |

A quartz cuvette containing an encapsulated hydrogen peroxide solution was radiated with UV light of various wavelengths. The top of the cuvette was covered with a balloon. The balloon collected the oxygen gas which was formed by the photodecomposition of hydrogen peroxide. The set up is shown in the Figurel.

The baseline value for all the examples is no oxygen gas generated which is demonstrated by a failure of the balloon to inflate.

An aqueous solution of Hydrogen peroxide was encapsulated in silica Dimethicone silylate by mixing the ingredients in the below table in an osterizer 12 speed blender for 2 minutes until a free flowing powder is formed.

The encapsulated powder of hydrogen peroxide was incorporated in an anhydrous antiperspirant stick. (data in Table 2 below)

TABLE 2

| Serial # | CTFA Name | % |
|---|---|---|
| 1 | Silica Dimethicone Silylate | 25.00 |
| 2 | Hydrogen peroxide (35%) | 20.00 |
| 3 | Water | 55.00 |
| | Total | 100.00 |

Activation of Hydrogen Peroxide Solution without antiperspirant active or encapsulation at a concentration 7.0%. Data in Table 3 below.

TABLE 3

| | | Amount of | | | Oxygen |
|---|---|---|---|---|---|
| Wave length | Distance | sample | | Duration | generated |
| 240 nm | 2 inches | 0.6339 | gm | 2 hrs. | yes |
| 380 nm | 1 inch | 1 | ml | 7 hrs. | yes |
| 265 nm | 1 inch | 1 | ml | 5 hrs. | yes |

Activation of Hydrogen peroxide Solution without antiperspirant active encapsulated with Silica Silylate. Data in Table 4 below.

TABLE 4

| | | Amount of | | | Oxygen |
|---|---|---|---|---|---|
| Wave length | Distance | sample | Duration | | generated |
| 405 nm | 2 inches | 0.2806 gm | 24 | hrs. | No |
| 355 nm | 2 inches | 0.3156 gm | 4 | hrs. | yes |
| 300 nm | 2 inches | 0.2406 gm | 2 | hrs. | yes |
| 325 nm | 2 inches | 0.2658 gm | 5 | hrs. | yes |
| 345 nm | 2 inches | 0.2931 gm | 6 | hrs. | yes |
| 380 nm | 2 inches | 0.1012 gm | 7 | hrs. | yes |
| 265 nm | 2 inches | 0.2751 gm | 4 | hrs. | yes |
| 240 nm | 2 inches | 0.2956 gm | 4 | hrs. | yes |
| 420 nm | 1 inch | 0.3711 gm | 24 | hrs. | No |
| 440 nm | 1 inch | 0.2955 gm | 24 | hrs. | No |
| 470 nm | 1 inch | 0.3036 gm | 24 | hrs. | No |
| 850 nm | 1 inch | 0.3021 gm | 24 | hrs | No |
| 940 nm | 1 inch | 0.2775 gm | 24 | hrs. | No |

Average mmoles of hydrogen peroxide in the above samples 0.62+/−0.04

Activation of Anhydrous Antiperspirant Product containing encapsulated Hydrogen peroxide Solution with Silica Silylate. Data in Table 5 below.

TABLE 5

| | | Amount of | | | Oxygen |
|---|---|---|---|---|---|
| Wave length | Distance | sample | Duration | | generated |
| 405 nm | 2 inches | 2.9712 gm | 23 | hrs. | No |
| 345 nm | 2 inches | 3.0744 gm | 24 | hrs. | yes |
| 355 nm | 2 inches | 2.9276 gm | 24 | hrs. | yes |
| 355 nm | 1 inch | 2.7152 gm | 24 | hrs. | yes |
| 300 nm | 2 inches | 3.5264 gm | 6 | hrs. | yes |
| 325 nm | 1 inch | 2.8115 gm | 24 | hrs. | yes |
| 265 nm | 1 inch | 2.8654 gm | 8 | hrs. | yes |
| 240 nm | 1 inch | 2.6912 gm | 8 | hrs. | yes |
| 380 nm | 2 inches | 3.3809 gm | 24 | hrs. | yes |
| 420 nm | 1 inch | 2.8005 gm | 24 | hrs | No |
| 440 nm | 1 inch | 2.9204 gm | 24 | hrs | No |
| 470 nm | 1 inch | 3.0522 gm. | 24 | hrs | No |
| 850 nm | 1 inch | 2.4232 gm | 24 | hrs. | No |
| 940 nm | 1 inch | 2.4243 gm | 24 | hrs. | No |

Average mmoles of hydrogen peroxide in the above samples 0.45+/−0.04

What is claimed is:

1. An antiperspirant/deodorant composition comprising: a hydrogen peroxide, wherein said composition is essentially free of aluminum cations or zirconium cations, and the composition is exposed to a light source having a wavelength range of >220 nm and <405 nm.

2. The composition of claim 1, wherein the light source is a light emitting diode.

3. The composition of claim 2, wherein the light emitting diode is contained in the cap of an antiperspirant container.

4. The composition of claim 1, wherein the hydrogen peroxide is encapsulated in silica dimethicone silylate.

5. The composition of claim 1, wherein the antiperspirant composition is in a form selected from the group consisting of a solid, a solid-like form, a liquid, and a semi-solid.

6. The composition of claim 1, wherein the wavelength is 355 nm.

7. The composition according to claim 1 wherein the percentage of perspiration reduction provided by said composition is greater than a placebo control absent said hydrogen peroxide exposed to a light source having a wavelength range of >220 nm and <405 nm, aluminum cations, and zirconium cations.

8. The composition according to claim 1 that further comprises: an absorbent, deodorant agent, antimicrobial, dye or colorant, emollient, moisturizer, fragrance, perfume, volatile oil, emulsifier, humectant, diluent, bulking agent, distributing agents, rheology agent, glyceride oil, silica, hair growth inhibitor, pharmaceutical active, UV active, preservative, surfactant, solvent, wash-off aid, and combinations thereof.

9. A method of activating hydrogen peroxide with a light source having a wavelength range of ≧220 nm and <405 nm in an antiperspirant/deodorant composition, wherein said composition is essentially free of aluminum cations or zirconium cations.

10. The method of claim 9, wherein the light source is a light emitting diode.

11. The method of claim 9, wherein the light emitting diode is contained in the cap of an antiperspirant container.

12. The method of claim 9, wherein the hydrogen peroxide is encapsulated in silica dimethicone silylate.

13. The method of claim 9, wherein the antiperspirant composition is in a form selected from the group consisting of a solid, a solid-like form, a liquid, and a semi-solid.

14. The method of claim 9, wherein the wavelength is 355 nm.

15. The composition according to claim 9 wherein the percentage of perspiration reduction provided by said composition is greater than a placebo control absent said hydrogen peroxide exposed to a light source having a wavelength range of >220 nm and <405 nm., aluminum cations, and zirconium cations.

16. The composition according to claim 9 that further comprises: an antiperspirant active, absorbent, deodorant agent, antimicrobial, dye or colorant, emollient, moisturizer, fragrance, perfume, volatile oil, emulsifier, humectant, diluent, bulking agent, distributing agents, rheology agent, glyceride oil, silica, hair growth inhibitor, pharmaceutical active, UV active, preservative, surfactant, solvent, wash-off aid, and combinations thereof.

17. The composition of claim 1 further comprising antiperspirant actives from natural sources.

18. The composition of claim 1 further comprising deodorant actives.

19. A container comprising a base for holding an antiperspirant/deodorant composition and a cap covering the base wherein the cap contains a light source on the internal side of the cap and the external side of the cap contains a switch controlling the light source.
